(19) United States
(12) Patent Application Publication
(10) Pub. No.: US 2026/0263784 A1
(43) Pub. Date: Sep. 10, 2026

(54) Adaptor for Percutaneous Anchoring of a Wire Lead Component of an Electrical Nerve Stimulation Assembly

(71) Applicant: Amitabh Goel, Lakewood Ranch, FL (US)

(72) Inventor: Amitabh Goel, Lakewood Ranch, FL (US)

(21) Appl. No.: 19/666,673

(22) Filed: May 4, 2026

(51) Int. Cl. A61N 1/05

(52) U.S. Cl. CPC A61N 1/0558

(57) ABSTRACT

An adaptor for percutaneous anchoring of a wire lead component of an electrical nerve stimulation assembly, the adaptor incorporating a left jaw having a proximal end, a distal end, and right side; a right jaw having a proximal end, a distal end, and a left side; a hinge interconnecting the proximal ends of the left and right jaws; at least a first lateral channel extending through the left and right jaws; and longitudinal channels for holding the wire lead component between the left and right jaws; the adapter incorporating at least a first screw received within the at least first lateral channel; the adapter incorporating a second longitudinally extending channel opening toward the at least first longitudinally extending channel, and incorporating a plurality of friction ridges, wherein each friction ridge extends into one of the longitudinally extending channels.

### FIELD OF THE INVENTION

The instant invention relates to assemblies for electric pulse suppression of pain, such assemblies being of the type which percutaneously extend an electrical wire lead and electrode head combination along a patient's spinal canal (or into electrical communication with one of the patient's peripheral spinal nerves), such lead and electrode head extension placing the electrode head at a targeted position in close proximity with the subject nerve. More particularly, the instant invention relates to fasteners and adaptors which anchor the wire lead component of such assemblies for resisting subsequent slippage and accidental displacement of the wire lead and electrode head.

### BACKGROUND OF THE INVENTION

The human spinal cord and peripheral spinal nerves include separate and discrete nerve pathways which transmit signals, including pain signals, to the brain from the body, and from the body to the brain. To achieve pain suppression via targeted electrical pulses directed to such nerves, a small electrode bearing head, configured either as a narrow cylinder or a flat wafer, may be extended to and placed at a selected position over the surface of the dura mater which covers a targeted nerve. Upon effecting a precisely targeted placement of such electrode head, electrical pulses emanating from the electrodes may effectively suppress the transmission of the signals which are responsible for neural transmission of the subject's pain. Such precise extension and placement of the electrode head may beneficially interfere with the spinal cord's transmission or peripheral nerve's transmission of pain sensations, and without energizing or interfering with the function of adjacent neural signal transmission tracks. Accordingly, such precise electrode head placement and electric pulse emitting operation may advantageously counteract the pain without impairing other nerve functions.

To introduce and place such an electrode head at a precisely determined location over the patient's spinal cord or over a peripheral nerve's dura mater, a physician or surgeon may percutaneously insert a hollow bored Tuohy needle at a location overlying the targeted nerve. A 14 gauge Tuohy needle is commonly used. Where the patient's spinal cord is targeted, the Tuohy needle may engage the patient's back in a manner which causes the needle's cutting point to pierce and traverse epidermis, dermis, and hypodermis layers of the patient's skin. The lancing action of the Tuohy needle may subsequently traverse the thoracolumbar facia tissues which cover the patient's back muscles, and may further traverse underlying muscle layers which directly overlie the patient's spine.

A further lancing insertion of the Tuohy needle may pierce interspinous ligaments spanning between spinal processes of the patient's vertebra, allowing the pointed end of the needle to enter the epidural space within the patient's spinal canal. Upon such Tuohy needle insertion, the hollow output end of the needle is positioned adjacent to the spinal cord. Such Tuohy needle insertion advantageously establishes a hollow bored pathway from the patient's exterior to and into the epidural space which overlies the patient's spinal cord dura mater. Such needle established pathway provides the surgeon access to the patient's spinal canal at a selected wire lead insertion site along the spinal cord without piercing the spinal cord's protective dura mater covering.

Following use of the Tuohy needle to establish a spinal canal access pathway, the surgeon may thread through the Tuohy needle in a distal direction the electrically conductive wire lead including the electrode head mounted at the lead's distal or longitudinal end. Further threading of the wire lead out of the needle's lancing point and distally through, for example, the patient's spinal canal is facilitated by a stainless steel stylet component within the wire lead. Such leads commonly include a hollow central bore which slidably receives a wire configured stainless steel stylet component which functions as a stiffening stay. The temporary presence of such stylet component within the wire lead allows a proximal end pushing force applied at an exterior proximal end of the wire lead to distally move and position the distally mounted electrode head without inducing “S” curving buckling of the wire lead.

By precisely threading, advancing, and positioning of such wire lead, the electrode head may be properly positioned for emissions of pain suppressing electrical pulses at and through a targeted track within the patient's spinal cord. Testing electrical pulses combined with reports of sensations made by the patient during the electrode head insertion procedure may assist the surgeon in establishing the desired precisely located position of the electrode head.

Following the precise placement of the electrode head, the stylet is rearwardly (i.e. proximally) slidably extracted from the wire lead. Thereafter, the Tuohy needle is commonly rearwardly withdrawn over the electrically conductive wire lead. Thereafter, the wire lead is desirably anchored upon back tissues which dorsally overlie the wire lead's point of entry into the spinal canal. Anchoring of the wire lead is typically needed because slight movements or slippage of the lead wire and electrode head may nullify the electrode head's ability to suppress the targeted pain sensations. A wire lead and electrode head migration as small as 1 mm may disrupt the function of the electrical stimulation assembly.

Where the targeted nerve is the patient's spinal cord, anchoring of the lead wire is commonly established at a zone or area underlying the patient's epidermis and dermis and immediately overlying the thoracolumbar facia covering of the patient's back musculature, through which the lead is traversing. Wire lead anchoring may be required at and upon other tissue sites remote from the spine which overlie a targeted peripheral spinal nerve.

Commonly known wire lead anchoring adaptors which are utilized for securing electrical stimulation lead wires against slippage are commonly installed with difficulty, and such adaptors commonly insecurely grasp and hold the wire lead. Such adaptors also are subject to slippage following surgical implantation.

Such commonly known tube or sleeve configured adaptors present extreme difficulties in surgical implantation, requiring that the adaptor be installed via threading the adaptor over the extreme proximal end of the wire lead prior to attachment of a battery powered electrical stimulation unit.

Since the tube or sleeve configured adaptors are configured for a slidable threading installation over the wire lead, such adaptors also commonly insecurely hold the wire lead in place upon surgical implantation.

Commonly known wire lead anchoring adaptors are incapable of securely clamping and holding the wire lead without distorting the circular cross-sectional shape of the lead's interior steel stylet receiving channel. Such channel distortion undesirably causes the wire lead clamping force to simultaneously clamp and fixedly position the stainless steel stylet within the wire lead. Accordingly, such anchors prevent a distally directed extraction of the stylet while the wire lead is anchored by the adaptor.

Utilization of known types of wire lead anchoring adaptors commonly requires that the wire lead stiffening stylet be proximally withdrawn from the wire lead prior to fixation and anchoring of the adaptor. Such preliminary stylet removal is often required because anchoring steps and procedures associated with of known adaptors may undesirably fix and seize the stylet within the lead's stylet receiving channel, preventing a subsequent removal of the stylet. Where the stiffening stylet is removed prior to performance of adaptor anchoring steps, the wire lead and electrode head stabilizing functions of the stylet are not performed during anchoring of the adaptor. Such known anchoring adaptors increase the risk of electrode head slippage and misalignment by requiring a preliminary or premature extraction of the stylet component.

The instant inventive adaptor for percutaneous anchoring of a wire lead component of an electrical nerve stimulation assembly solves or ameliorates such problems by configuring the adaptor to include a “C” configuration, and by incorporating within the adaptor transverse screw receiving channels which allow a transverse direction of installation with respect to the longitudinal extension of the wire lead. The screw clamped “C” configuration of the inventive adaptor is desirably consistent with the continued presence of components mounted proximally upon the wire lead, such as a wire lead guiding Tuohy needle. The inventive adaptor additionally provides superior holding forces against the wire lead by providing the screw and lateral channel combinations. The inventive adaptor additionally performs a function of clamping a received wire lead without distorting the circular cross-sectional shape of the lead. By avoiding such distortion, the inventive adaptor effectively clamps the wire lead without clamping and fixing a stainless steel stylet component within the lead, thereby allowing a sliding extraction of the stylet while the adaptor performs its lead clamping function.

### BRIEF SUMMARY OF THE INVENTION

The instant inventive adaptor for percutaneous anchoring of a wire lead component of an electrical nerve stimulation assembly preferably comprises left and right jaws, each jaw having proximal and distal ends, and each jaw having left and right sides.

A further structural component of the instant inventive adaptor comprises a hinge which spans laterally between and interconnects the proximal ends of the left and right jaw components. In the preferred embodiment, the left and right jaw components are composed of a durable plastic material, and the hinge comprises a living hinge which is formed wholly or integrally with the left and right jaws.

A further structural component of the instant inventive adaptor comprises at least a first transverse or lateral channel which extends through both the left jaw and the right jaw. The combination of the left and right jaw components and the interconnecting hinge forms a “C” configuration whose downwardly extending arms define a throat between the “C” arms. In the preferred embodiment, the at least first lateral channel comprises a pair of segments, each segment among the pair extending through one of the jaws, wherein said each segment has an inner end which communicates with the “C” configuration's throat.

Means for holding the electric lead wire within the throat and between the left and right arms are preferably provided. In a preferred embodiment, such means comprise left and right longitudinally extending channels which respectively extend leftwardly and rightwardly into the left and right jaws, each such channel opening toward and communicating with the adaptor's throat. In the preferred embodiment, the left and right longitudinally extending channels, when pivoted toward each other, together form a circular channel which matches the circular cross-sectional profile of the wire lead.

Such wire leads having circular cross-sections commonly incorporate a central hollow bore which slidably receives a wire configured stainless steel stylet. While such stylet resides within such hollow bore, pushing forces exerted by a surgeon against an exterior proximal end of the wire lead may effectively distally move the lead's distal electrode head. Accordingly, such steel stylet allows, for example, the surgeon to push the electrode head along a patient's spinal canal without inducing “S” curving buckling of the wire lead.

The circular channel which is preferably formed by the adaptor's left and right longitudinally extending channels advantageously allows the inwardly facing walls of such channels to press against and hold the wire lead without distorting the circular cross-sectional shape of the lead's interior st stylet ay receiving channel. By preventing such channel distortion, the instant inventive adaptor is able to securely clamp and hold the wire lead without clamping and fixing the lead's st stylet ay. Accordingly, the preferred embodiment of the inventive adaptor allows for a sliding proximally directed extraction of the stylet out of the wire lead while the wire lead remains securely clamped and anchored by the adaptor. The instant adaptor's ability to anchor the wire lead while preserving the capacity for sliding extraction of the stylet allows the stylet to assist with stabilizing the electrode head while the adaptor is utilized for lead clamping and anchoring. Since sliding extraction of the stylet is preserved, the step of removal of the stylet may be advantageously performed after completion of wire lead anchoring.

The holding means component may suitably, though less desirably, alternatively comprise a single longitudinally extending channel which extends either into the left jaw or into the right jaw. Where the holding means comprise one or more longitudinally extending channels, longitudinally extending ridges or fingers are preferably provided for enhanced frictional contact with and holding of a channel received wire lead.

In use of the instant inventive adaptor, a surgeon may position the lower mouth opening of the “C” configuration formed throat component directly above the lead wire. Thereafter, the surgeon may manually extend the lead wire upwardly into the interior of the adaptor so that the lead wire extends longitudinally within and along the throat. Such upward lead wire extension preferably positions the lead wire so that its left and right sides laterally overlie the preferably provided longitudinally extending channels.

Thereafter, the surgeon may extend a screw laterally into the at least first lateral channel. Where the at least first lateral channel presents pre-formed internal helical threads, such screw may engage such threads. Alternatively, the screw may constitute a self-tapping screw which cuts its own internal helical threads within the adaptor's jaws as it is screw-driven laterally through the at least first lateral channel.

In a preferred embodiment, the at least first lateral channel comprises a pair of channel segments wherein one of such channel segments extends laterally through one of the adaptor's jaws, and wherein the other channel segment extends through the opposite jaw. Where such segmented configuration is adopted, one of the segments may be sized for a lateral sliding receipt with the opposite channel having a smaller diameter for screw thread engagement. Such arrangement of channel segment diameters advantageously allows the larger diameter segment which initially slidably receives the screw to perform an initial screw holding function. While the screw is held in place by the larger diameter segment, a driver tool may be applied to the screw for successive threaded engagement with the smaller diameter screw.

Screw tightening of such screw advantageously draws the adaptor's jaws together against the lead wire, securely clamping and anchoring the lead wire. The preferably cross-sectionally circular channel which is formed by the left an right longitudinally channels advantageously allows the inner surfaces of such channels to compressively hold the wire lead without distorting the lead into a flattened oval on non-circular shape. By avoiding such distortion of the wire lead, the circular cross-sectional shape of the wire lead's interior stylet receiving channel is preserved, advantageously preventing any frictional binding or clamping against the lead's temporary interior stiffening stylet. Accordingly, the instant inventive adaptor is able to securely clamp the wire lead without interfering with a subsequent sliding extraction of the lead's stylet.

Following such screw actuated clamping, the surgeon may extend sutures about and/or through the adaptor to securely hold both the adaptor and the lead wire clamped within the adaptor at the surgical implant location.

In a preferred embodiment of the instant inventive adaptor, multiple lateral channels are provided within and through the adaptor's jaws, such multiple lateral channels allowing the insertions of multiple screws, and providing at least a first passage for through extensions of adaptor anchoring sutures. Following screw actuated clamping and sutured anchoring of the adaptor, the surgeon may distally slidably extract the lead's stylet. Thereafter, the Tuohy needle may be withdrawn, and the incision at the implantation site may be surgically closed.

Accordingly, objects of the instant invention include the provision of an adaptor for percutaneous anchoring of a wire lead component of an electrical nerve stimulation assembly which incorporates structures as described above, and which arranges those structures in relation to each other in manners described above for the performance and achievement of beneficial functions as described above.

Other and further objects, benefits, and advantages of the instant invention will become known to those skilled in the art upon review of the detailed description which follows, and upon review of the appended drawings.

### BRIEF DESCRIPTION OF DRAWINGS

FIG. 1 is a perspective view of a suitable embodiment of the instant inventive adaptor for percutaneous anchoring of a wire lead component of an electrical nerve stimulation assembly.

FIG. 2 is a sectional view as indicated in FIG. 1.

FIG. 3 re-depicts the structure of FIG. 2, the view of FIG. 3 additionally showing, in exploded views, screw and wire lead components.

FIG. 4 presents an alternate configuration of the structures of FIG. 3.

FIG. 5 presents a further alternate configuration of the structures of FIG. 3.

FIG. 6 re-depicts the structure of FIG. 1, the view of FIG. 6 showing installed wire lead, screw, and suture components.

FIG. 7 is a sectional view as indicated in FIG. 6.

FIG. 8 presents an alternate configuration of the structure of FIG. 2.

FIG. 9 re-depicts the structure of FIG. 8, the view showing arms-engaging forceps and a wire lead.

FIG. 10 presents a further alternate configuration of the structure of FIG. 2.

FIG. 11 presents a further alternate configuration of the structure of FIG. 2.

FIG. 12 presents an alternate configuration of the structure of FIG. 3.

FIG. 13 is an alternate sectional view, as indicated in FIG. 1

FIG. 14 presents an alternate configuration of the structure of FIG. 13.

FIG. 15 is a right-side view of the structure of FIG. 6, the view showing the adaptor surgically installed within a tissue implantation site overlying a targeted nerve.

Structures of the drawings which are identified by reference numerals having a suffix “A”, “B”, “C”, “D”, or “E” are configured similarly with similarly numbered structures (i.e. structures identified by a suffix-less number) appearing in other drawings.

### DETAILED DESCRIPTION OF THE INVENTION

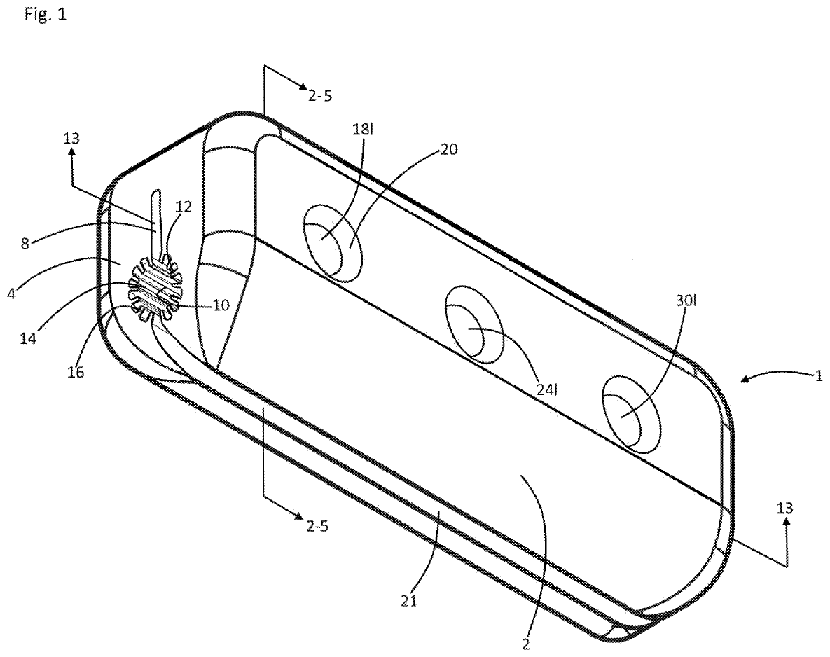

Referring now to the drawings, and in particular to FIG. 1, a preferred embodiment of the instant inventive adaptor for percutaneous anchoring of a wire lead component of an electrical nerve stimulation assembly is referred to generally by reference arrow 1. The adaptor 1 preferably comprises a downwardly or distally extending left jaw 2 and a downwardly or distally extending right jaw 4. The adaptor 1 further comprises a hinge 6 which spans laterally between the upper or proximal ends of the left and right jaws.

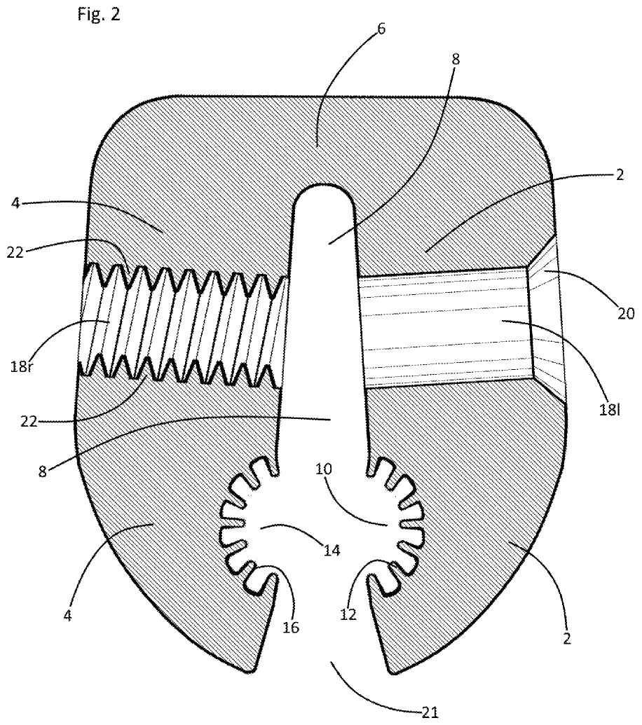

In a preferred embodiment, the left and right jaws 2 and 4 are composed of a durable plastic material, and the hinge 6 preferably comprises a plastic living hinge which is formed integrally or wholly the jaws' upper or proximal ends. As shown in FIG. 2, the adaptor's combination of left and right jaws 2 and 4 and the upper living hinge 6 form a downwardly facing or opening “C” configuration whose arms inwardly define a throat space 8. The living hinge 6 constitutes a ceiling or upper wall of the throat 6 while the open space 21 at the lower end of the throat 8 constitutes the throat's mouth.

The instant inventive adaptor preferably comprises at least a first laterally extending channel. As shown in FIG. 2, such channel includes a left segment 18l which extends laterally through the left jaw 2, and a right segment 18r which mirroringly extends through the adaptor's right jaw 4. Each of such channel segments 18l and 18r has an outer opening, and has an inner opening which communicates with the adaptor's throat 8.

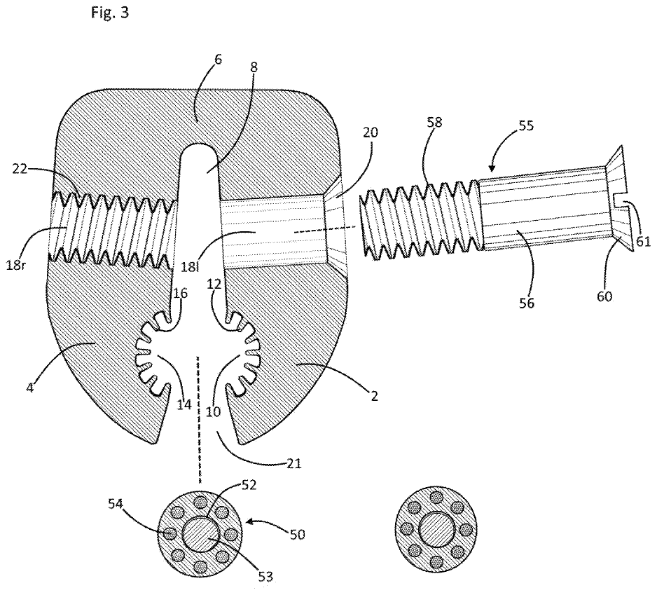

In use of the instant inventive adaptor, referring further to FIG. 3, the living hinge 6 may be specially adapted to include a plastic memory characteristic which slightly splays the left and right jaws 2 and 4 away from each other. Such normal jaw splay may laterally widen the adaptor's longitudinally extending mouth 21 a distance sufficient to allow an a surgeon's manual upward insertion of a wire lead 50 through the mouth 21 and into the adaptor's throat 8. Such wire lead 50 may commonly include and incorporate an interior electrical conductors 54, which are arrayed about an interior circular stylet receiving channel 52. A wire configured stainless steel stylet 53 is commonly slidably extended into and along the channel 52. Such channel and stylet combination may advantageously resist “S” curving or buckling of the wire lead upon extension via pushing against the lead's proximal end.

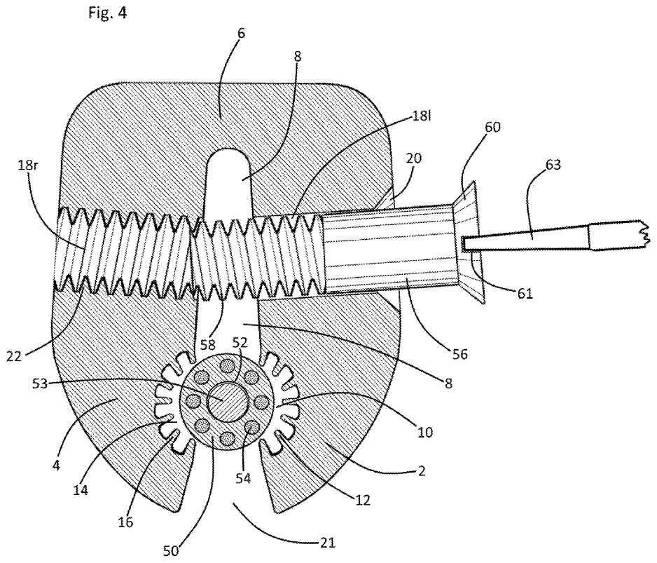

Referring further to FIG. 4, upon such upward insertion of the wire lead 50 through the mouth 21, the wire lead 50 may be nestingly received within and retained by left and right longitudinally extending channels 10 and 14. Such channels constitute preferred wire lead holding means which enhance frictional holding contact between the left and right jaws 2 and 4 and the left and right sides of the wire lead 50. The facing channels 10 and 14 together form a circular channel within the adaptor, such circular channel preferably matching the circular cross-sectional profile of the wire lead 50.

As a result of the circular match of the circular channel 10,14 and the cross-section of the wire lead 50, inwardly directed clamping forces exerted by the channels 10 and 14 against the wire lead allow the lead to retain its circular shape. Such shape retention advantageously avoids distortion or flattening of the lead's interior channel 52, advantageously avoiding translation of adaptor's lead clamping force into a wire stylet clamping force. Accordingly, the circular channel forming jaw channels 10 and 15 dually perform a clamping function and a clamp resisting function, the clamping function being directed to the wire lead and the clamp resisting function being directed to the lead's temporarily installed wire stylet 53.

The adaptor's holding means may additionally comprise a plurality of longitudinally extending friction ridges or friction fingers 12 and 16 extend inwardly from the concave outer walls of the left and right channels 10 and 14. There such ridges or fingers are provided, their extreme inner ends preferably for the circular wire lead receiving circular channel as described above.

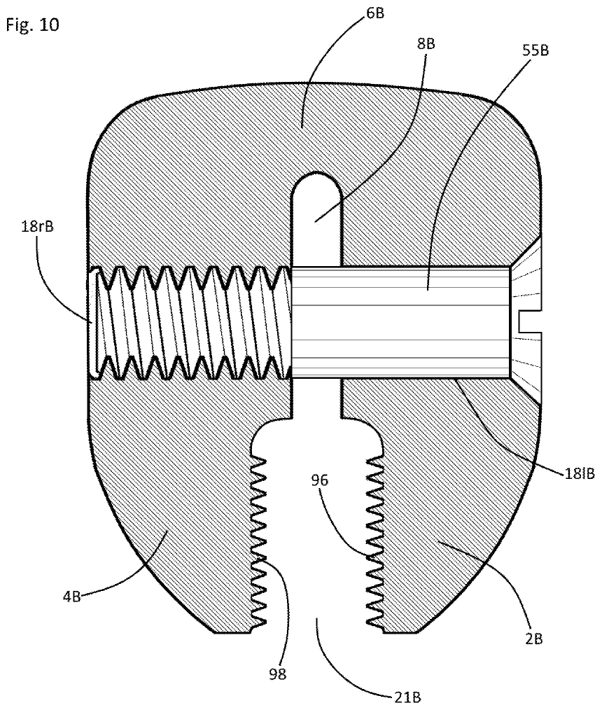

Referring further to FIG. 10, all structures identified by a reference numeral having a suffix “B” are configured similarly with similarly numbered structures appearing in other figures. In the configuration of FIG. 10, alternatively configured wire lead holding means comprise the respective right and left walls of the left and right jaws 2B and 4B in combination with knurling or “V” configured friction ridges 96 and 98. In the FIG. 10 configuration, the wire lead holding means perform their holding function without any provision of a longitudinally extending lead receiving channel. The FIG. 10 configuration may be less desirable than the FIGS. 1-5 configuration due to imposed distortion of the stylet receiving channel 52 and resultant fixing of the stylet 53 within the lead.

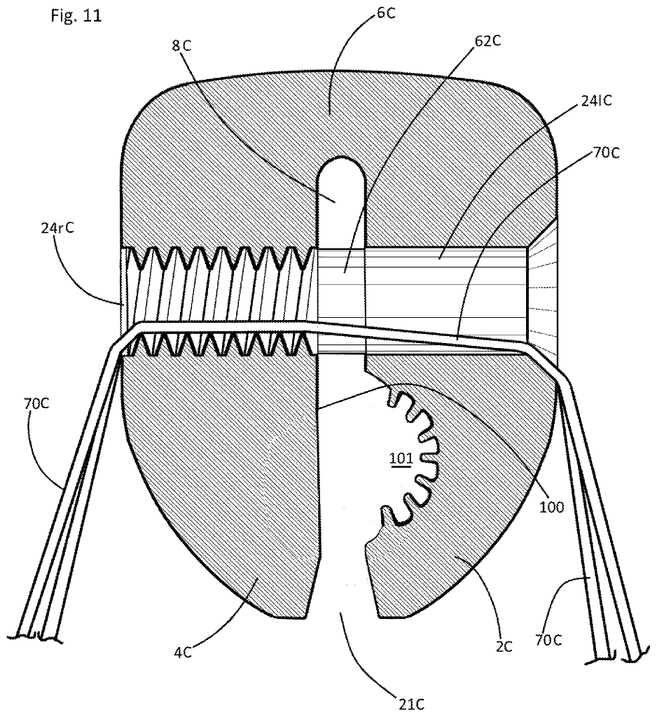

In the further alternate configuration of FIG. 11, the holding means may suitably, though less desirably, comprises a single longitudinally extending channel 101 which faces a flat jaw wall 100, and which nestingly receives a lateral side or aspect of a wire lead. Further alternatively, the opposing jaw faces may comprise a single “V” valley or a facing pair or “V” valleys (not depicted within views).

Referring to FIG. 4, following an upward receipt of the wire lead 50 within the left and right longitudinally extending and circular channel forming channels 10 and 14, or alternatively within the single channel 101 or between frictional “V” ridges 96 and 98, a screw 55 may be rightwardly inserted into and through the leftward opening of the left lateral channel segment 181, such rightward screw insertion continuing until the rightward end of the screw 55 enters the leftward opening of the right lateral channel segment 18r.

The depicted preferred smooth walled configuration of the left lateral channel segment 181 facilitates such rightward sliding insertion and right lateral channel segment entry. The ability of the smooth-walled lateral channel segment 181 to initially slidably receive a surgeon's manually effected rightward extension of the screw 55 promotes efficiencies in the surgeon's assembly and use of the inventive adaptor. For example, while the surgeon's left hand is occupied with other tasks, the surgeon may utilize his right hand to rightwardly insert the screw 55 into the channel segment 181. Thereafter, the surgeon may release the screw, allowing the channel segment 181 to continue to hold the screw at a position wherein the rightward end of the screw directly laterally overlies leftward opening of the right lateral channel segment 18r. While the screw 55 is so held by the adaptor, the surgeon may freely access a driver tool 63, and may engage the tool with the slot 61 within the screw's head 60.

Following the sliding insertion of the screw 55 into the position indicated in FIG. 4, and following engagement of the blade of the screw driver 63 with the head 60 of the screw, the surgeon may turn the screwdriver clockwise, thereby turning the screw 55 clockwise. Rotation of the screw threadedly engages the screw's male helical threads 58 with female helical threads 22 which line the wall of the right lateral channel segment 18r.

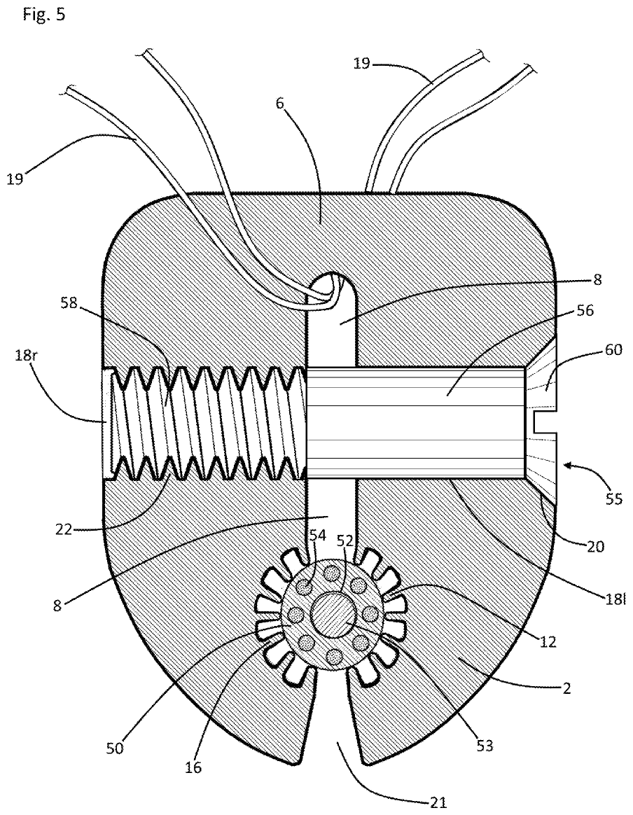

Referring further to FIG. 5, continued screwdriver actuated turning of the screw 55 allows screw threads 58 and the screw head 60 to draw the left and right jaws 2 and 4 toward each other in a contracting motion, such drawing action being effected through engagement of the male screw threads 58 with female screw threads 22 and through engagement of the screw's head 60 with the left jaw's screw head receiving recess 20.

Such screw actuated contracting action drives the left and right jaws 2 and 4 inwardly toward each other and against the wire lead 50 as indicated in FIG. 5. Compressive and frictional holding of the wire lead 50 is enhanced by finger ridges 12 and 16 which extend inwardly from the walls of the left and right channels 10 and 14. The preferred circularly extending array of the inner ends of the ridges 12 and 16 advantageously prevents distortion of the circular cross-sectional shape of the wire lead, preserving the circular shape of the leads stylet receiving channel 52, and preventing clamping and binding of the temporarily received stylet 53. Upon complete assembly as indicated in FIG. 5, the wire lead 50 is securely held within the throat 8 of the adaptor 1 while the stylet 53 remains free to move slidably within channel 52.

Referring further to FIG. 5, where the instant inventive adaptor presents and utilizes a single screw 55 and a single laterally extending channel 18l, 18r the adaptor's longitudinally extending and downwardly opening throat 8 may be suitably utilized as a passage channel for receipt of adaptor anchoring sutures 19. Such sutures 19 necessarily overlie the wire lead 50, and preferably overlie the medial portion of the screw 55 which bridges across the throat 8. Ends of the sutures 19 may be extended, through use of attached suture needles (not depicted within views), through underlying tissues for secure anchoring of both the adaptor 1 and the wire lead 50.

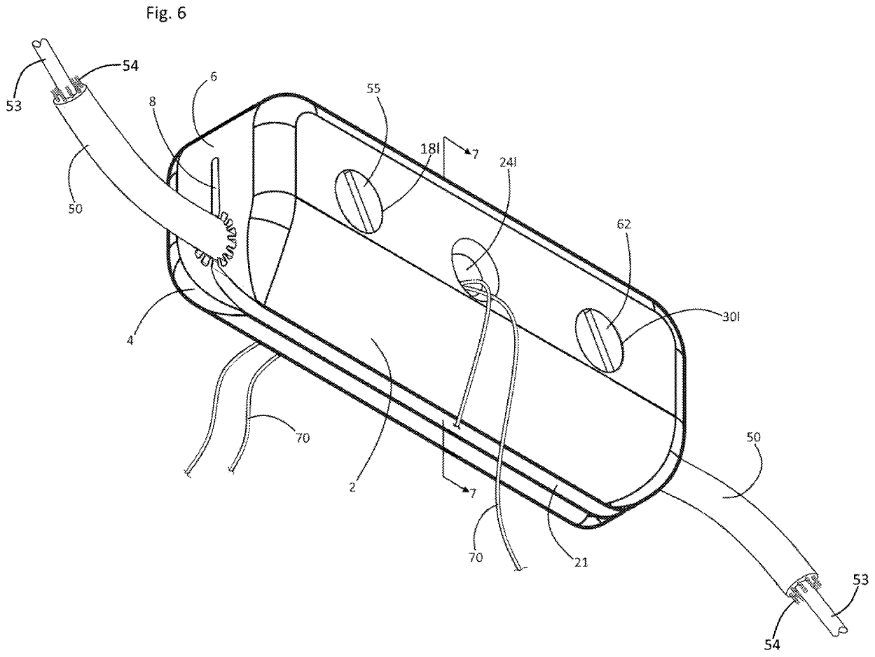

As shown in FIGS. 1 and 6, the adaptor 1 preferably includes further or additional laterally extending channels, 24l,24r and 30l,30r. Each of such additional channels is preferably configured substantially identically with the at least first laterally extending channel 181, 18r, such additional channels suitably having smooth-walled leftward segments and having internally helically threaded rightward segments. In such embodiment, a pair of screws 55 and 62 may be respectively inserted into and engaged within channels 18l, 18r, and 30l,30r while the adaptor's middle or third channel 24l,24r remains open. Such open middle channel 24l,24r may be advantageously utilized for lateral extensions and receipts of adaptor anchoring sutures 70.

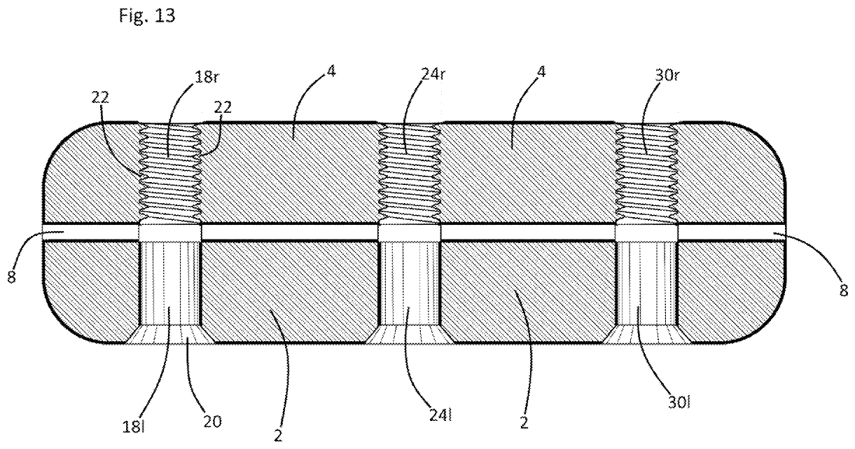

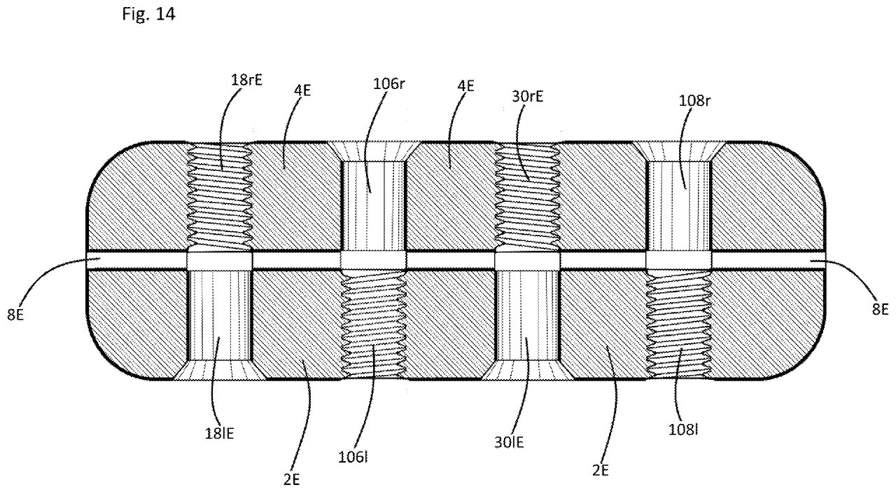

Referring simultaneously to FIGS. 1, 6, 13, and 14, it may be seen that versatility in modes of assembly and use of the adaptor may be achieved by presenting a plurality of lateral channels wherein some channels have their smooth-walled segments oriented leftwardly, and some of the smooth-walled segments are oriented rightwardly. In the alternate embodiment of FIG. 14, the orientations of lateral channels 106l, 106r and 108l, 108r are reversed in relation to channels 18lE, 18eE and 30lE,30rE. By presenting the oppositely oriented lateral channels, the adaptor may advantageously include an ambidextrous characteristic which allows a surgeon to choose among leftward and rightward extensions of clamping screws, and to choose the direction of application of a driver tool to the adaptor.

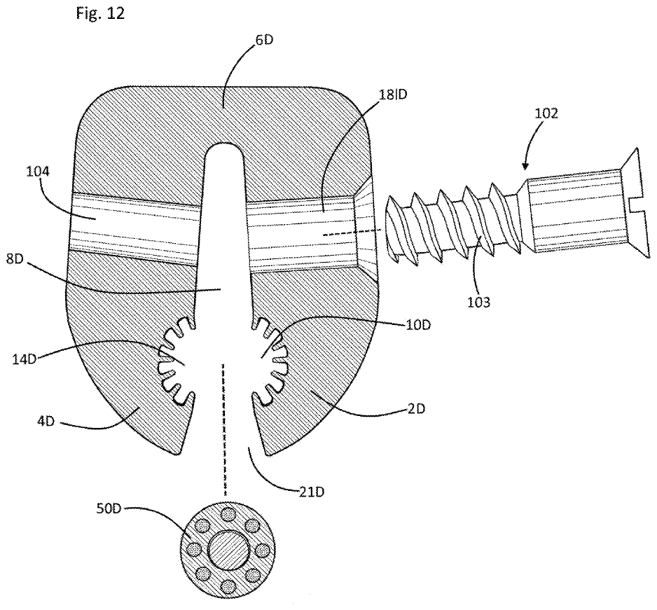

Referring simultaneously to FIGS. 3 and 12, all structures identified by a reference numeral having a suffix “D” are configured similarly with similarly numbered structures appearing in other figures. In the FIG. 12 alternative configuration, the screws 102 include self-tapping helical threads 103. Upon a rightward sliding extension of such screw 102 into lateral channel segment 18lD, the self-tapping helical threads 103 may enter and engage the wall of a narrower smooth-walled right channel segment 104. In the FIG. 12 configuration, the threads of such alternative screw 102 threadedly engage the smooth wall of channel segment 104 to draw jaws 2D and 4D toward each other against wire lead 50D.

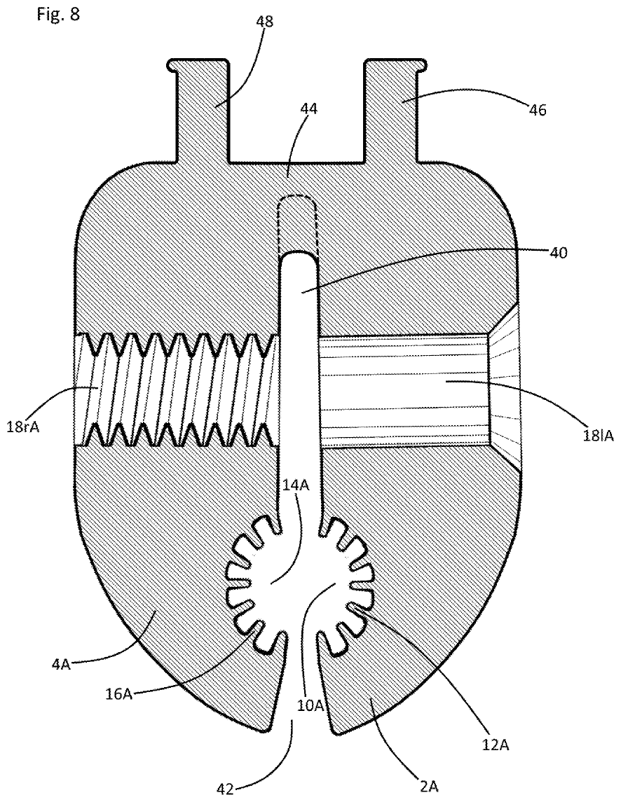

Drawing FIGS. 1, 2, and 3 represent and depict a plastic memory characteristic or feature of the adaptor 1 wherein the living hinge component 6 normally holds the left and right jaws 2 and 4 in a slightly laterally splayed configuration. Accordingly, in the FIGS. 1-3 configuration the left and right jaws 2 and 4 may initially exert little if any holding force against the wire lead 50 upon the lead's upward insertion into the adaptor's throat 8. Referring further to FIG. 8, in which reference numerals having a suffix “A” are configured similarly with similarly numbered structures appearing in other figures, an alternatively formed living hinge 44 includes a plastic memory characteristic which normally holds the left and right jaws 2A and 4A at a contracted or retracted position. Upon receipt of a wire lead within the channel space defined by left and right longitudinally extending channels 10A and 14A, the plastic memory of such alternate living hinge 44 normally inwardly compresses the jaws 2A and 4A against such lead wire. In the FIG. 8 configuration, the adaptor functions as a spring clip or “C” clip which normally performs a wire lead holding function.

However, in the FIG. 8 plastic memory living hinge configuration, the normally contracted mouth opening 42 at the lower end of the adaptor's central throat 40 may resist an upward insertion of a lead wire. To allow the lead wire to be upwardly inserted through such normally narrow mouth 42 of the alternately formed adaptor of FIG. 8, left and right lever arms 46 and 48 may be fixedly attached to and may extend upwardly from the upper or proximal ends of the left and right jaws 2A and 4A.

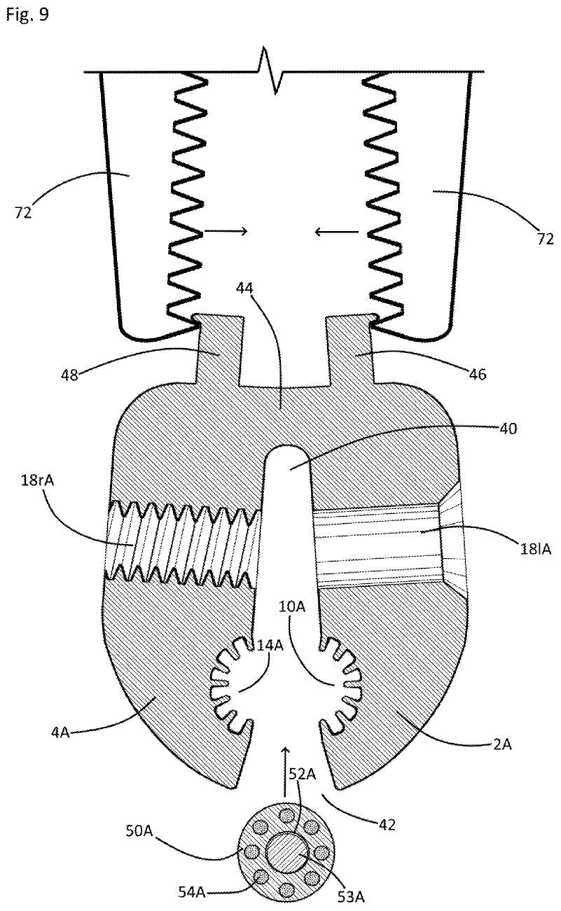

Referring further simultaneously to FIG. 9, paired jaws 72 of forceps or pliers may engage the lever arms 46. Upon application of inwardly directed compressive forces, as indicated by arrows drawn upon FIG. 9, the lever arms 46 and 48 may be driven toward each other, thereby pivoting the left and right jaws 2A and 4A away from each other, in a splaying action about the living hinge 44. To effect such splaying action, the living hinge 44 functions a fulcrum in relation to the lever arms 46 and 48.

Upon an upward insertion of the wire lead 50A into and through the widened opening 42 and into throat 40, the forceps' jaws 72 may be released from the lever arms 46 and 48. Such release allows the plastic memory of the living hinge 44 to counter-pivot the left and right jaws 2A and 4A toward each other, advantageously compressively holding the wire lead 50A within the left and right channels 10A and 14A.

Dashed lines drawn upon FIG. 8 represent an alternative vertical narrowing of the living hinge 44. By narrowing such hinge, use of the forceps 72 may be obviated, such narrowing allowing a surgeon to effectively use fingertip pressure to manipulate the lever arms 46 and 48.

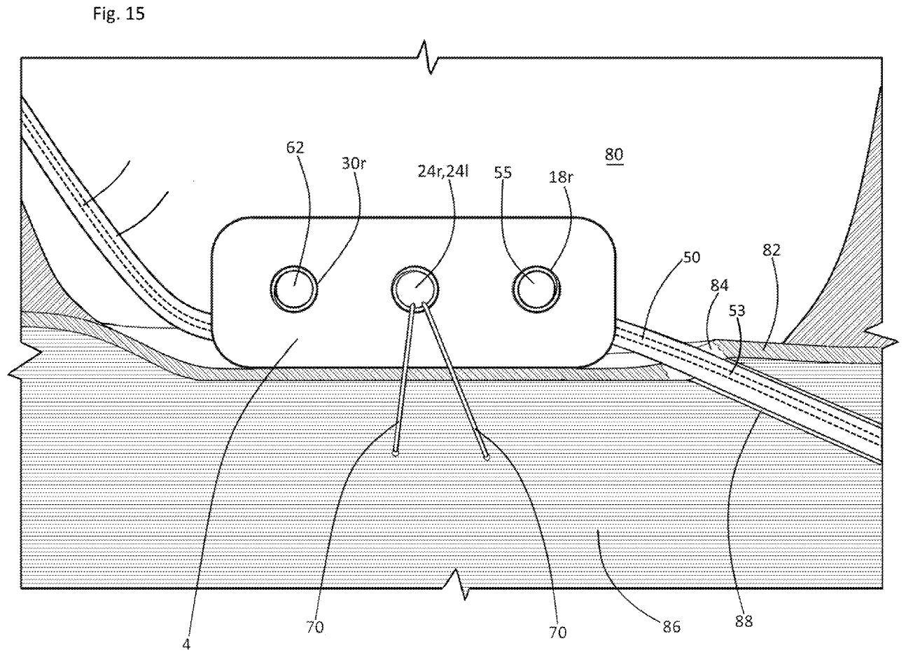

Referring to the assembled and deployed configuration of FIG. 15, a surgical incision 80 has been pre-established within a patient's body, the incision traversing epidermis, dermis, and hypodermis layers of the patient's skin at the chosen site of electrical stimulator implantation. An underlying muscle fascia layer 52 may form a floor of such incision 80, and the lead wire 50 may be preliminarily extended via a lancing insertion of a Tuohy needle (not depicted within view) through an aperture 84 within the fascia 82. Such needle may further extend to form an angled channel 88 within underlying musculature 86.

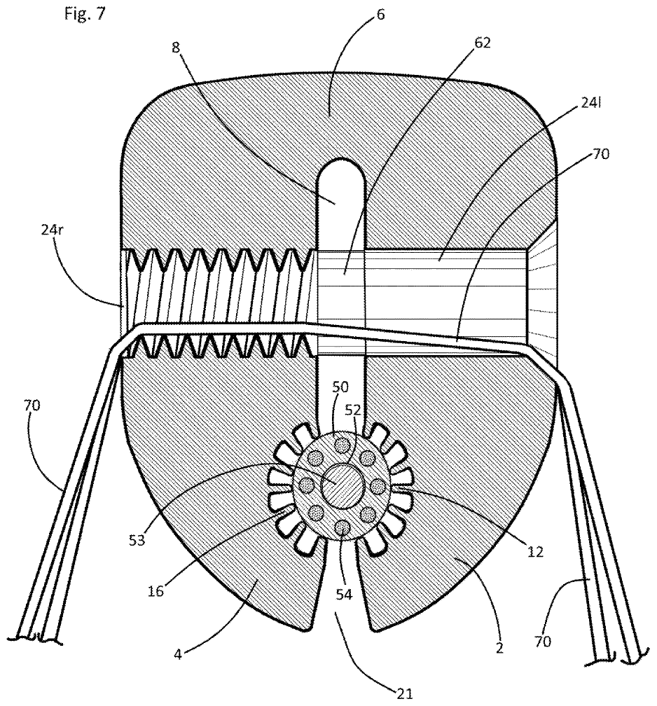

Referring further to FIGS. 6 and 7, the wire lead 50 may then be inserted upwardly through mouth 21 until the left and right sides of the lead partially nest within the adaptor's left and right longitudinally extending channels 10 and 14. Thereafter, screws 55 and 62 may be extended into and tightened within lateral channels 181, 18r, and 301, 30r. Upon tightening of such screws, the wire lead 50 becomes securely clamped within the throat 8 of the adaptor 1. As a result of the preferred circular configuration of the channels 10 and 14, such clamping force against the wire lead 50 leaves the lead's interior stylet 53 free to slidably move within its interior channel.

Thereafter, the surgeon may extend sutures 70 laterally through lateral channel segments 24l,24r to securely anchor both the adaptor 1 and the wire lead 50 at its desired implantation location within incision 80.

The instant inventive adaptor advantageously allows the continued presence of the stylet 53 within the wire lead 50 while the surgeon manipulates the adaptor for receipt and positioning of the wire lead, while the surgeon further manipulates the adaptor for insertion of screws to clamp the adaptor against the wire lead, and while the surgeon further manipulates the adaptor for extension of sutures through the lateral channel segments 24l,24r. Each of such surgeon performed adaptor manipulations imposes some risk of slippage or displacement of the distal extension of the wire lead and its distal electrode head. The continued presence of the stylet 53 within wire lead during performance of such manipulations advantageously reduces the risk of such slippage and displacement.

Following clamping and suturing of the adaptor and wire lead within the implantation site incision 80, the stylet 53 may be slidably withdrawn in the proximal direction from the wire lead. Thereafter, the Tuohy needle may be slidably withdrawn and the incision 80 may be surgically closed.

While the principles of the invention have been made clear in the above illustrative embodiment, those skilled in the art may make modifications in the structure, arrangement, portions and components of the invention's assembly without departing from those principles. Accordingly, it is intended that the description and drawings be interpreted as illustrative and not in the limiting sense, and that the invention be given a scope at least commensurate with the appended claims.

1. An adaptor for percutaneous anchoring of a wire lead component of an electrical nerve stimulation assembly, said adaptor comprising:
a. a left jaw having a proximal end, a distal end, and right side;
b. a right jaw having a proximal end, a distal end, and a left side;
c. a hinge interconnecting the proximal ends of the left and right jaws;
d. at least a first lateral channel extending through the left and right jaws; and
e. means for holding the wire lead component between the left and right jaws.

2. The adaptor of claim 1, further comprising at least a first screw received within the at least first lateral channel.

3. The adaptor of claim 2, wherein the means for holding the wire lead component between the left and right jaws comprise at least a first longitudinally extending channel, said channel opening either leftwardly at the left side of the right jaw or opening rightwardly at the right side of the left jaw.

4. The adaptor of claim 3, comprising a second longitudinally extending channel opening toward the at least first longitudinally extending channel, wherein the at least first and second longitudinally extending channels form a circular channel.

5. The adaptor of claim 4 wherein the left and right jaws are composed of plastic, and wherein the hinge comprises a living hinge.

6. The adaptor of claim 5 further comprising a plurality of longitudinally extending ridges, each longitudinally extending ridge extending into the circular channel.

7. The adaptor of claim 6 comprising a second lateral channel extending through the left and right jaws, and further comprising a second screw received within the second lateral channel.

8. The adaptor of claim 7 wherein each lateral channel is internally helically threaded.

9. The adaptor of claim 7 further comprising a third lateral channel, wherein the third lateral channel extends through the left and right jaws.

10. The adaptor of claim 9, further comprising sutures extending through the third lateral channel.

11. The adaptor of claim 6 wherein the left and right jaws are moveable between a splayed position for a receipt within the longitudinally extending channels of the wire lead component, and a contracted position wherein the longitudinally extending ridges impinge against said received wire lead component.

12. The adaptor of claim 11 wherein the at least first lateral channel has internal helical threads, and wherein the at least first screw engages said threads to draw the left and right jaws toward their contracted positions.

13. The adaptor of claim 12 wherein the living hinge has plastic memory which normally positions the left and right jaws at their splayed positions.

14. The adaptor of claim 12 wherein the living hinge has plastic memory which normally positions the left and right jaws at their contracted positions.

15. The adaptor of claim 14 further comprising left and right lever arms, said arms being respectively fixedly attached to and extending from the left and right jaws' proximal ends.

16. The adaptor of claim 1 wherein the at least first lateral channel is between the hinge and the left and right jaws' distal ends.

17. The adaptor of claim 16, further comprising a throat between the left and right jaws, wherein the at least first screw bridges across said throat.

18. The adaptor of claim 17 wherein the at least first lateral channel comprises left and right channel segments, and wherein each of said channel segments communicates with the throat.

19. The adaptor of claim 18 wherein one of the at least first channel's channel segments is internally helically threaded, and wherein the at least first channel's other of the channel segment is smooth walled.

20. The adaptor of claim 19 wherein the at least first screw has a smooth walled head end, and wherein said screw has a helically threaded end.
